(19) United States
(12) Patent Application Publication
(10) Pub. No.: US 2026/0024662 A1
(43) Pub. Date: Jan. 22, 2026

(54) Comprehensive AI-Driven Digital Health Platform for Personalized Care and Device Management

(71) Applicant: OHANNES OHANNESSIAN, Round Rock, TX (US)

(72) Inventor: OHANNES OHANNESSIAN, Round Rock, TX (US)

(21) Appl. No.: 18/773,951

(22) Filed: Jul. 16, 2024

(51) Int. Cl. G16H 50/30; G16H 10/60; G16H 30/40

(52) U.S. Cl. CPC G16H 50/30; G16H 10/60; G16H 30/40

(57) ABSTRACT

The invention provides a comprehensive AI-driven digital health platform for personalized mental health, dental care, and self-health management. It integrates computer vision, NLP, and ML to generate actionable insights, personalized recommendations, and a personal health score. The platform facilitates secure communication between users and healthcare providers, offers device management, and utilizes blockchain for transparent transactions. It also includes a data science platform for ML model development and deployment, and a pooling system to aggregate outputs from multiple AI/ML models for enhanced accuracy. The invention aims to improve health outcomes and patient-centered care through advanced technologies and personalized guidance.

### BACKGROUND

### Field of the Invention

The present invention relates to the field of digital health management, specifically to a platform that integrates artificial intelligence (AI), machine learning (ML), and data analytics to provide personalized solutions for mental health, dental care, and self-health management.

### Description of Related Art

Digital health technologies are transforming the healthcare landscape by providing innovative solutions to address various health challenges. In particular, mobile health (mHealth) applications and platforms have emerged as powerful tools for remote patient monitoring, disease management, and health promotion.

One such example is disclosed in WO2017075496A1, which describes a personal mobile health management system. The system includes a mobile application running on a mobile device with wireless connectivity, personal health records stored on the device, a database system to store the records, an alert management module to alert caregivers based on unique alert levels, a permission management module to control access to the records, a software module for integration between the mobile application and sensors to receive sensor data, and a data processing module to process the received sensor data, update the personal health record, and evaluate health updates to determine if an alert is needed.

While this system provides a foundation for mobile health management, there remain significant challenges and opportunities for improvement in the areas of mental health, dental care, and self-health management.

Nearly one in five adults in the U.S. experience mental illness annually, with limited access to care and stigma being major barriers. In dental health, over 47 million Americans face a shortage of dental care professionals, and poor dental health can lead to systemic health issues. Many individuals also struggle with monitoring and managing their overall health and wellness.

Existing digital health platforms often lack the advanced capabilities needed to address these challenges effectively. They may provide basic data aggregation but fail to deliver actionable insights or personalized recommendations. Additionally, user privacy and data control are not always prioritized, which can hinder trust and adoption.

Therefore, there is a need for a comprehensive digital health platform that leverages cutting-edge technologies such as AI, ML, and computer vision to provide personalized, user-centric solutions for mental health, dental care, and self-health management. Such a platform should prioritize user privacy, generate actionable insights, and be tailored to the unique needs of these critical healthcare areas.

### SUMMARY

The present invention addresses the need for a comprehensive digital health platform that leverages artificial intelligence (AI), machine learning (ML), and data analytics to provide personalized solutions for mental health, dental care, and self-health management. The platform comprises a processor and a memory storing instructions that, when executed, cause the platform to perform several key functions.

A user interface is provided to receive user input and display personalized health information. A data integration module collects and stores health data from various sources, including user input, medical devices, electronic health records, wearable devices, and remote patient monitoring systems. The platform employs a computer vision module that utilizes object recognition, feature extraction, and image segmentation techniques to analyze medical images and videos, identifying abnormalities and assisting in diagnosis. This module also uses deep learning algorithms to detect and classify skin conditions, retinal abnormalities, and dental issues from user-provided images.

A natural language processing (NLP) module interprets and extracts insights from user input, medical records, and questionnaire responses. It also analyzes sentiment and emotion in user input to assess mental well-being and provide personalized recommendations for stress management and mental health support.

At the core of the platform is an AI engine comprising machine learning models trained on health data to generate personalized health insights, predict disease risks, and provide tailored recommendations. The AI engine includes a predictive analytics module that utilizes machine learning algorithms to forecast potential health risks and enable early interventions based on user health data and trends. A reinforcement learning model continuously adapts and personalizes health recommendations based on user feedback and outcomes.

The platform generates a personal health score based on the collected health data and AI-generated insights, providing personalized alerts, notifications, and recommendations to users based on their health status and predicted risks. It also facilitates communication between users and healthcare providers for improved patient-centered care through a secure messaging system and an application programming interface (API) for integration with third-party applications and services.

Additional features include a chatbot interface powered by the NLP module and AI engine for engaging users in natural language conversations, providing health guidance, and triaging user inquiries to appropriate healthcare resources. The user interface presents health insights and recommendations using interactive data visualizations, while a gamification module employs game design elements and rewards to incentivize user engagement and promote healthy behaviors.

The invention also provides a computer-implemented method for AI-driven mental health assessment and recommendation. The method involves receiving user input, collecting mental health data from various sources, analyzing the data using NLP, computer vision, and machine learning techniques, generating a personalized mental health profile, providing tailored recommendations, monitoring user progress, updating the profile and recommendations based on new data, and facilitating secure communication with mental health professionals.

The invention also incorporates a device management platform that utilizes token-based API requests for enhanced security, integrates advanced data ingestion and analysis, and provides augmented reality (AR) services. It includes a comprehensive data science platform for machine learning model development and deployment, a pooling system to aggregate outputs from multiple AI/ML models for enhanced accuracy, and a blockchain-based platform for secure and transparent transactions of devices and maintenance tickets.

The invention also incorporates a device management platform that utilizes token-based API requests for enhanced security, integrates advanced data ingestion and analysis, and provides augmented reality (AR) services. It includes a comprehensive data science platform for machine learning model development and deployment, a pooling system to aggregate outputs from multiple AI/ML models for enhanced accuracy, and a blockchain-based platform for secure and transparent transactions of devices and maintenance tickets.

In summary, the present invention provides a comprehensive, AI-driven digital health platform that addresses the unique challenges and opportunities in mental health, dental care, and self-health management. By leveraging advanced technologies and prioritizing user privacy, the platform generates actionable insights and personalized recommendations to improve health outcomes and patient-centered care.

Additional features and advantages of the invention will be set forth in the description which follows, and in part will be obvious from the description, or may be learned by the practice of the invention. These and other features of the present invention will become more fully apparent from the following description, or may be learned by the practice of the invention as set forth hereinafter.

### BRIEF DESCRIPTION OF THE DRAWINGS

The various embodiments of the present invention, which will become more apparent as the description proceeds, are described in the following detailed description in conjunction with the accompanying drawings, in which:

FIG. 1 illustrates a system diagram of an embodiment of a digital health self-assessment platform for collecting, analyzing, and providing personalized health insights and recommendations.

FIG. 2 illustrates a user interface navigation diagram for an embodiment of a digital health self-assessment platform.

FIG. 3 illustrates a user interface navigation diagram for an embodiment of a platform for monitoring oral and physiological health data.

FIG. 4 illustrates another embodiment of a system diagram for the digital health self-assessment platform and their interactions.

FIG. 5 illustrates a flow diagram of an embodiment of a computer-implemented method for AI-driven mental health assessment and recommendation using the integrated platform architecture.

### DETAILED DESCRIPTION

In the following detailed description of the preferred embodiments, reference is made to the accompanying drawings, which form a part hereof and show, by way of illustration, specific embodiments in which the invention may be practiced. It is to be understood that other embodiments may be used and structural or logical changes may be made without departing from the scope of the present invention. The following detailed description, therefore, is not to be taken in a limiting sense, and the scope of the present invention is defined by the appended claims.

The following description is provided as an enabling teaching of the present systems, and/or methods in its best, currently known aspect. To this end, those skilled in the relevant art will recognize and appreciate that many changes can be made to the various aspects of the present systems described herein, while still obtaining the beneficial results of the present disclosure. It will also be apparent that some of the desired benefits of the present disclosure can be obtained by selecting some of the features of the present disclosure without utilizing other features.

Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

The terms “a” and “an” and “the” and similar references used in the context of describing a particular embodiment of the present invention (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein.

All systems described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application. Thus, for example, reference to “an element” can include two or more such elements unless the context indicates otherwise.

As used herein, the terms “optional” or “optionally” mean that the subsequently described event or circumstance can or cannot occur, and that the description includes instances where said event or circumstance occurs and instances where it does not.

The word or as used herein means any one member of a particular list and also includes any combination of members of that list. Further, one should note that conditional language, such as, among others, “can,” “could,” “might”, or “may” unless specifically stated otherwise, or otherwise understood within the context as used, is generally intended to convey that certain aspects include, while other aspects do not include, certain features, elements and/or steps. Thus, such conditional language is not generally intended to imply that features, elements and/or steps are in any way required for one or more particular aspects or that one or more particular aspects necessarily include logic for deciding, with or without user input or prompting, whether these features, elements and/or steps are included or are to be performed in any particular aspect.

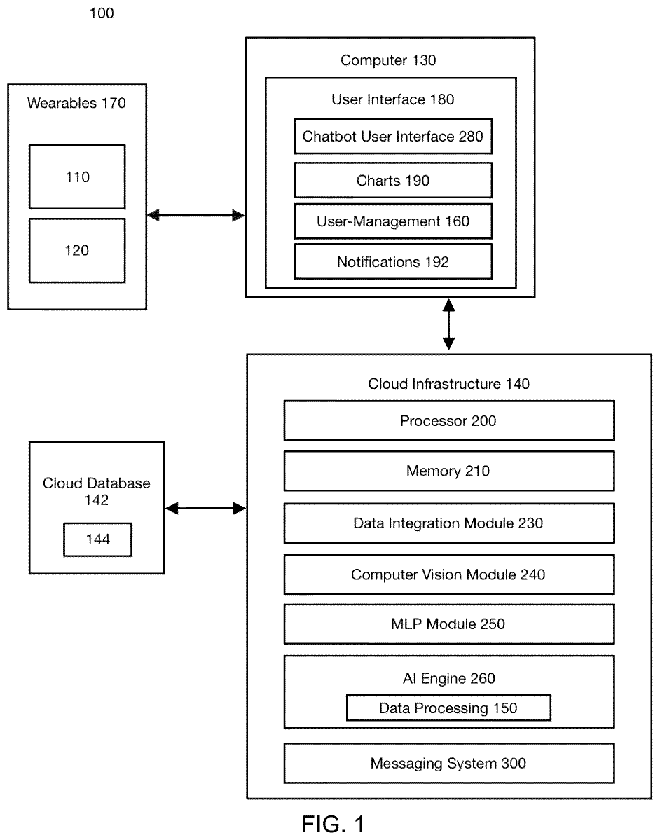

FIG. 1 illustrates an embodiment of a system diagram of a digital health self-assessment platform 100 for collecting, analyzing, and providing personalized health insights and recommendations.

Wearables 170 are integral to the platform 100. The wearables 170 are customized and distributed based on individual oral composition, collecting data such as ID, pressure, acidity, accelerometer readings, GPS location, and temperature. In some embodiments the software is customizable for deeper analysis.

In some embodiments the platform 100 comprises a plurality of input sources configured to collect health data from a user. The input sources include oral-specific sensors 110, such as dental YLM sensors, oral YLM guards, and smart dental retainers, adapted to collect data on tooth positioning, teeth friction pressure, and mouth activity. The platform 100 further includes general physiological sensors 120 configured to measure parameters such as temperature, pressure, acidity, humidity, heart rate, oxygen saturation, blood pressure, and hydration levels.

The sensor data is transmitted to a smartphone or computer 130 for initial processing and connectivity to a cloud infrastructure 140. The cloud infrastructure 140 comprises a secure cloud database 142 configured to store the processed data and cloud applications 144 that enable further data processing and analytics.

User management 160 is handled by the platform 100, wherein users register by providing personal, medical, and insurance information via the user interface 180. Upon confirmation, they receive a registration ID and can select between standard and premium packages. The premium package offers unlimited data storage and advanced analytics per user and disease type within an organization.

In some embodiments the user interface 180 provides account management features, including password reset and login/logout functionality. It offers tools such as the Merck Manual, medicine search, forms to PDF conversion, and ECG. Services available through the user interface 180 include dental AI, sensor connectivity, skin disease detection, optical image processing, self-check, and mental health support. AI assistants, music, and video recommendations are also available.

The platform 100 is configured to generate output and alerts 192 in the form of SMS, email, and other notifications. Users can access analytics, historical data, and tracking information. Detected conditions trigger corresponding alerts, such as dehydration, teeth misalignment, or mouth overheating.

The computer vision module 240, executed by the one or more processors 200, employs advanced deep learning algorithms to analyze medical images and videos. These deep learning algorithms, such as convolutional neural networks (CNNs), are trained on large datasets of labeled medical images to learn features and patterns associated with various skin conditions, retinal abnormalities, and dental issues. The training process involves iteratively adjusting the weights and biases of the neural network layers to minimize the difference between predicted and actual labels, using techniques such as backpropagation and gradient descent.

Once trained, the computer vision module 240 applies these learned features to new, unseen images provided by users. The module preprocesses the images by resizing, normalizing, and augmenting them to ensure consistency and robustness. It then uses techniques such as object recognition to detect and localize specific anatomical structures or abnormalities, feature extraction to identify key visual characteristics, and image segmentation to delineate boundaries between healthy and affected tissues.

The computer vision module 240 outputs a detailed analysis of the medical images, including the presence and severity of any detected conditions, along with visualizations highlighting the relevant regions of interest. This information is then integrated with other user health data by the AI engine 260 to generate comprehensive personalized health insights and recommendations.

The natural language processing (NLP) module 250, executed by the one or more processors 200, employs advanced techniques such as tokenization, named entity recognition, and sentiment analysis to interpret and extract insights from unstructured text data. Tokenization involves breaking down user input, medical records, and questionnaire responses into individual words or subwords, which are then processed to remove stop words and perform stemming or lemmatization.

Named entity recognition is used to identify and classify key medical concepts, such as symptoms, diagnoses, medications, and treatments, within the text data. The NLP module 250 is trained on large corpora of medical text, annotated with relevant named entities, to learn patterns and context associated with these concepts.

Sentiment analysis is applied to assess the emotional tone and mental well-being of users based on their language use. The NLP module 250 is trained on datasets of text labeled with sentiment scores, allowing it to learn associations between language patterns and emotional states. By analyzing sentiment and emotion in user input, the module can provide personalized recommendations for stress management and mental health support.

The extracted insights and structured data from the NLP module 250 are then fed into the AI engine 260 to inform personalized health predictions and recommendations, in conjunction with data from other sources such as the computer vision module 240 and user health records.

In some embodiments the stored data undergoes further extensive data processing 150, to generate real-time health diagnosis, detect issues and generate a personal health score.

The platform 100 provides an application programming interface (API) 270, executed by the one or more processors 200, that enable integration with third-party applications and services.

The one or more processors 200 execute instructions stored in the memory 210 to generate a personal health score based on the collected health data and AI-generated insights. It is further configured to provide personalized alerts, notifications, and recommendations to users based on their health status and predicted risks. The platform 100 also facilitates communication between users and healthcare providers for improved patient-centered care.

A chatbot interface 280, powered by the NLP module 250 and AI engine 260, is configured to engage users in natural language conversations, provide health guidance, and triage user inquiries to appropriate healthcare resources.

The user interface 180, executed by the one or more processors 200, is configured to present health insights and recommendations using interactive data visualizations 290, including charts, graphs, and infographics, to facilitate user understanding and engagement.

A secure messaging system 300, executed by the one or more processors 200, is configured to enable encrypted communication between users and healthcare providers, allowing for the exchange of health data, test results, and treatment plans.

A gamification module 310, executed by the one or more processors 200, is configured to employ game design elements and rewards to incentivize user engagement, promote healthy behaviors, and encourage adherence to self-assessment routines.

In operation, the digital health self-assessment platform 100 collects health data from a plurality of input sources, including oral-specific sensors 110 and general physiological sensors 120. The collected data is transmitted to a smartphone or computer 130 for initial processing and connectivity to the cloud infrastructure 140, where it is stored in the secure cloud database 142 by the data integration module 230.

The stored data undergoes extensive data processing 150, including reporting, scoring, and insight analysis performed by the AI engine 260. The computer vision module 240, analyzes medical images and videos to identify abnormalities, while the NLP module 250, executed by the one or more processors 200, interprets user input and medical records.

Users interact with the platform 100 through the user interface 180, which provides account management features, tools, and services. The chatbot interface 280, executed by the one or more processors 200, engages users in natural language conversations and provides health guidance.

Based on the collected health data and AI-generated insights, the one or more processors 200 executes instructions stored in the memory 210 to generate a personal health score and provide personalized alerts, notifications, and recommendations to users. The gamification module 310, executed by the one or more processors 200, incentivizes user engagement and promotes healthy behaviors.

The platform 100 facilitates communication between users and healthcare providers through the secure messaging system 300, executed by the one or more processors 200, enabling the exchange of health data, test results, and treatment plans.

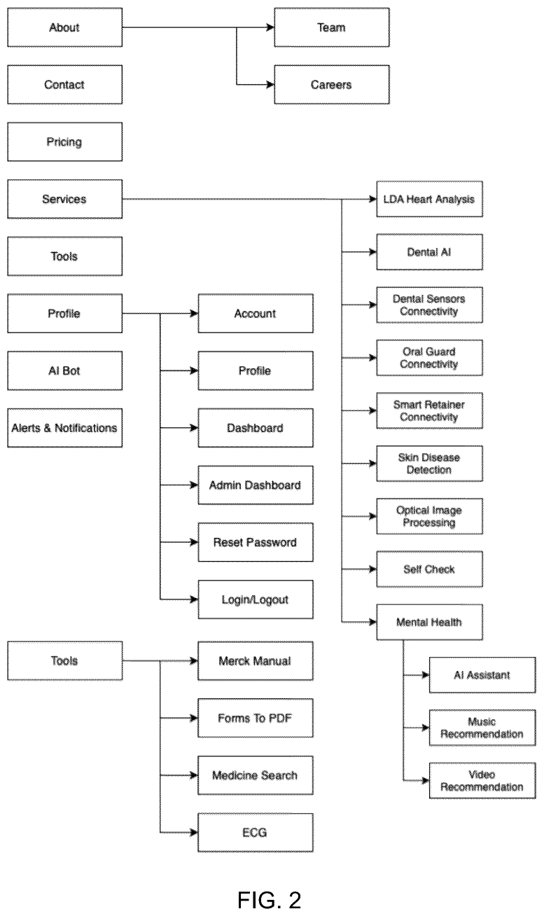

FIG. 2 illustrates a user interface navigation diagram for a digital health self-assessment platform 100. The user interface 180, 220 provides a hierarchical navigation structure that enables users to access various features and functionalities of the platform 100.

The main navigation menu includes top-level items such as Home, About, Contact, Pricing, Services, Tools, Profile, AI Bot, and Alerts & Notifications. The About section links to sub-pages containing information about the Team and Careers.

Under the Profile section, users can access their Account, view and edit their Profile, access their personal Dashboard, and, for administrators, access the Admin Dashboard. This section also includes options for users to Reset Password and Login/Logout.

The Tools section provides access to various resources, including the Merck Manual, a tool to convert Forms To PDF, a Medicine Search feature, and an ECG (Electrocardiogram) tool.

The Services section offers a range of analytical and AI-powered tools, such as LDA Heart Analysis, Dental AI, Dental Sensors Connectivity 110, Oral Guard Connectivity, Smart Retainer Connectivity, Skin Disease Detection, Optical Image Processing, Self Check, and Mental Health resources.

Within the Mental Health category, additional AI-powered features are available, including an AI Assistant, Music Recommendation, and Video Recommendation.

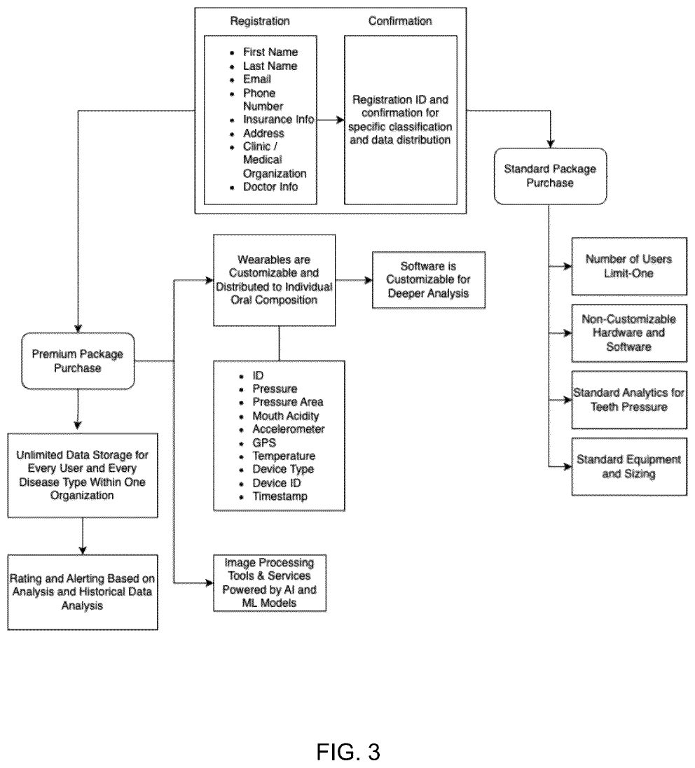

FIG. 3 illustrates an embodiment of a user interface navigation diagram for a platform 100 for monitoring oral and physiological health data. The user interface 180 guides the user through a registration process to collect relevant personal and medical information. The registration interface prompts the user to input their first name, last name, email address, phone number, insurance information, physical address, associated clinic or medical organization, and doctor information.

Upon completion of the registration process, the platform 100 generates a confirmation interface displaying a unique registration ID and confirming the user's specific classification and data distribution settings. As shown in FIG. 3, the user is then presented with a package selection interface offering two options: a Standard Package and a Premium Package.

In one embodiment, the Standard Package is limited to a single user and includes non-customizable hardware and software components 170, standard analytics for monitoring teeth pressure, and standard equipment and sizing. In contrast, the Premium Package offers unlimited data storage for every user and every disease type within one organization, as well as advanced rating and alerting features based on analysis of historical data.

Both packages include wearable devices equipped with oral-specific sensors 110 and general physiological sensors 120. The wearables are customizable and distributed to users based on their individual oral composition. The software components 170 are also customizable, thereby enabling deeper analysis of the collected data.

As depicted in FIG. 3, the wearable devices collect various data points, including a unique identifier (ID), pressure measurements, pressure area, mouth acidity levels, accelerometer data, GPS coordinates, temperature readings, device type and ID, and timestamps. This data is transmitted to a smartphone or computer 130 and then uploaded to a cloud infrastructure 140.

Within the cloud infrastructure 140, the data is stored in a secure cloud database 142 and processed by cloud applications 144 and data processing modules 150. According to an embodiment, the Premium Package includes additional image processing capabilities powered by artificial intelligence (AI) and machine learning (ML) models.

The processed data and insights are then presented to the user via the user interface 180, 220 on their smartphone or computer 130. The user interface includes output and alerts 190 configured to notify users of any significant findings or anomalies detected in their oral and physiological health data.

As shown in FIG. 3, the platform 100 is powered by a processor 200 and memory 210, wherein said processor and memory enable the execution of various modules and components, comprising a data integration module 230, computer vision module 240, natural language processing (NLP) module 250, and AI engine 260. An application programming interface (API) 270 facilitates communication and data exchange between the platform and external systems.

Optionally, additional features of the user interface may include a chatbot interface 280 for user support and inquiries, interactive data visualizations 290 for presenting insights and trends, a secure messaging system 300 for communication between users and healthcare providers, and a gamification module 310 to encourage user engagement and adherence to oral and physiological health monitoring practices.

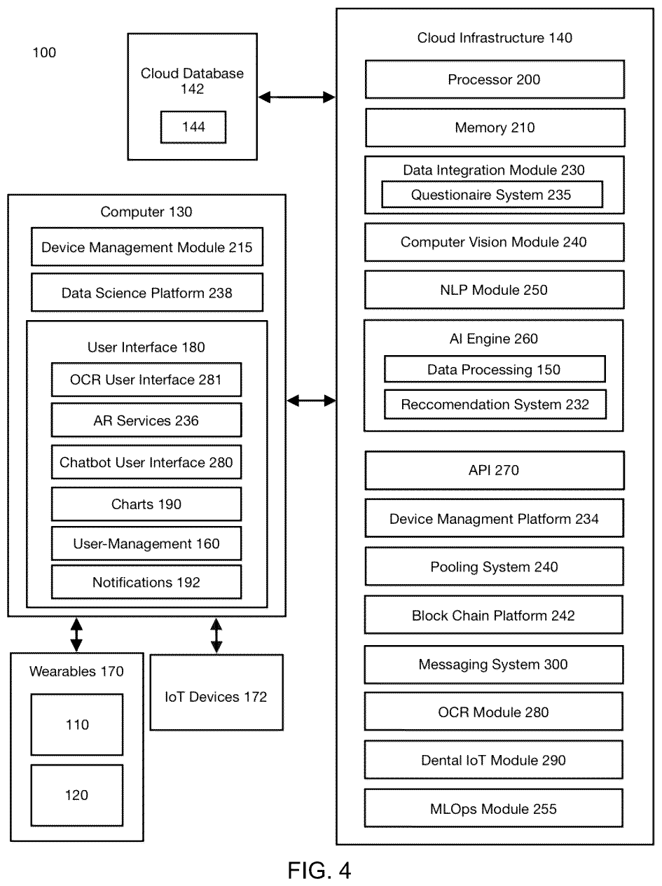

FIG. 4 illustrates another embodiment of a system diagram for the digital health self-assessment platform 100 and their interactions. As shown in FIG. 4, the digital health self-assessment platform 100 comprises one or more processors 200 and a memory 210 storing instructions. When executed by the one or more processors 200, said instructions cause the platform 100 to perform various functions and operations.

With reference to FIG. 4, the digital health self-assessment platform 100 provides a user interface 180 configured to receive user input and display personalized health information. The user interface 180 enables users to interact with the platform 100, input their health data, and receive tailored recommendations and insights.

In one embodiment, the platform 100 implements a data integration module 230 configured to collect and store health data from a plurality of sources, including user input, medical devices 110, and electronic health records 152. The data integration module 230 ensures seamless integration and interoperability of health data from various sources, thereby creating a comprehensive health profile for each user.

As depicted in FIG. 4, a computer vision module 240 is implemented in the platform 100, wherein said module is configured to analyze medical images and videos using object recognition, feature extraction, and image segmentation techniques. The computer vision module 240 identifies abnormalities and assists in diagnosis by processing and analyzing visual medical data.

In another embodiment, the platform 100 implements a natural language processing (NLP) module 250 configured to interpret and extract insights from user input, medical records, and questionnaire responses. The NLP module 250 enables the platform 100 to understand and process unstructured textual data, thereby enhancing the platform's ability to provide personalized recommendations and insights.

At the core of the digital health self-assessment platform 100 is an artificial intelligence (AI) engine 260 comprising machine learning models trained on health data. The AI engine 260 generates personalized health insights, predicts disease risks, and provides tailored recommendations based on the user's health profile and historical data.

Illustrated in FIG. 4 is the digital health self-assessment platform 100 providing an application programming interface (API) 270 configured to enable seamless integration with a plurality of third-party applications and services. The API 270 utilizes secure communication protocols, such as HTTPS and OAuth, to facilitate encrypted data exchange between the platform 100 and external systems. By exposing a well-documented set of endpoints and methods, the API 270 allows authorized third-party applications to securely access and exchange relevant health data with the platform 100, thereby expanding its functionality and reach. The API 270 implements rate limiting and throttling mechanisms to prevent abuse and ensure optimal performance.

According to an embodiment, the digital health self-assessment platform 100 further comprises a device management module 215 configured to integrate with and manage a plurality of wearable and non-wearable medical devices 170. The device management module 215 establishes secure connections with the medical devices 170 using protocols such as Bluetooth Low Energy (BLE), Wi-Fi, or cellular networks. It receives structured health data from the connected medical devices 170 at predefined intervals and stores the data in the secure cloud database 142 for further processing. Additionally, the device management module 215 transmits control signals and configuration settings to the medical devices 170, enabling remote management and customization of device behavior.

As shown in FIG. 4, the AI engine 260 includes a recommendation system 232 that generates profile-based recommendations, performs AI-based translation, conducts health risk assessments, triggers intelligent alerts, and enables smart enrollments based on user preferences and historical data.

The recommendation system 232 employs advanced machine learning algorithms, such as collaborative filtering and content-based filtering, to analyze user profiles, medical history, and behavioral patterns. By leveraging these insights, the recommendation system 232 delivers highly personalized and actionable recommendations to users via the user interface 180. The AI-based translation component of the recommendation system 232 facilitates multilingual communication and content adaptation, ensuring that users receive recommendations and insights in their preferred language.

In one embodiment, the digital health self-assessment platform 100 incorporates a device management platform 234 that enhances security and encryption through the use of token-based API requests. The device management platform 234 generates and validates unique access tokens for each connected medical device 170, ensuring that only authorized devices can transmit data to the platform 100.

Furthermore, the device management platform 234 integrates advanced data ingestion and analysis capabilities, enabling real-time processing of incoming health data and generation of actionable insights. These insights are presented to users through interactive visualizations and personalized reports within the user interface 180.

Additionally, the device management platform 234 provides augmented reality (AR) services 236 that blend virtual elements with the real-world environment, creating an immersive and engaging user experience. The AR services 236 leverage computer vision and image recognition techniques to overlay relevant health information, guidance, and animations onto the user's view through the user interface 180, enhancing understanding and adherence to health recommendations.

Optionally, the digital health self-assessment platform 100 includes a comprehensive data science platform 238 that supports the development, deployment, and management of machine learning models. The data science platform 238 provides a collaborative environment for data scientists, offering features such as ML model management, version control, and performance tracking. It includes a recommendation system that suggests optimal ML algorithms and hyperparameters based on the specific health assessment tasks at hand.

The data science platform 238 also incorporates advanced detection analysis capabilities, enabling the identification of anomalies, patterns, and trends within the health data. Seamless integration with popular data science tools, such as Jupyter Notebook and JupyterLab, allows data scientists to leverage existing workflows and libraries, thereby accelerating the development and deployment of sophisticated ML models.

Alternatively, the digital health self-assessment platform 100 incorporates a pooling system 240 that aggregates outputs from multiple AI and ML models, including those developed by third-party organizations and hosted on external databases. The pooling system 240 employs ensemble learning techniques, such as bagging and boosting, to combine the predictions of diverse models and generate more accurate and reliable outcomes. It dynamically weights the contributions of each model based on its historical performance and domain expertise, ensuring optimal results across various health assessment tasks. The pooling system 240 also incorporates advanced data fusion algorithms to seamlessly integrate outputs from heterogeneous data sources, such as structured EHR data, unstructured clinical notes, and real-time sensor data.

In one embodiment the digital health self-assessment platform 100 includes a blockchain-based platform 242 for buying and selling devices and maintenance tickets. The blockchain-based platform 242 features a marketplace module, blockchain ledger, smart contracts, user authentication, and a user interface. The blockchain ledger securely records transactions, while the smart contracts automate the buying and selling process, thereby ensuring transparency and trust.

In one embodiment this marketplace module is built on top of a permissioned blockchain network, such as Ethereum, which ensures the immutability and traceability of all transactions. The blockchain ledger maintains a tamper-proof record of device ownership, transfer history, and maintenance activities, providing a single source of truth for all stakeholders. Smart contracts, which are self-executing contracts with the terms of the agreement directly written into code, are employed to automate and enforce the buying and selling process. These smart contracts define the rules and conditions for device transactions, such as pricing, warranties, and delivery terms, and are automatically triggered when predefined conditions are met. The use of smart contracts eliminates the need for intermediaries, reduces transaction costs, and ensures the integrity and security of the transactions.

In some embodiments the blockchain-based platform 242 also incorporates robust user authentication mechanisms, such as multi-factor authentication and biometric verification, to prevent unauthorized access and protect user privacy. A user-friendly interface is provided, allowing users to easily navigate the marketplace, view device specifications and ratings, and initiate transactions using various payment methods, including cryptocurrencies. By leveraging the inherent benefits of blockchain technology, the blockchain-based platform 242 creates a trustworthy and efficient ecosystem for the exchange of medical devices and services within the digital health self-assessment platform 100.

In some embodiments, the instructions stored in the memory 210 further cause the platform 100 to generate a personal health score based on the collected health data and AI-generated insights. The platform 100 provides personalized alerts, notifications, and recommendations to users based on their health status and predicted risks via the output and alerts module 192, thereby empowering them to take proactive steps towards better health.

As further depicted in FIG. 4, the digital health self-assessment platform 100 incorporates an optical character recognition (OCR) module 280 configured to extract text from images of medical documents and forms. The OCR module 280 integrates an OCR library or API (e.g., Tesseract, Google Cloud Vision API) into the platform's backend. An OCR user interface component 281 is implemented to allow users to upload images of medical documents and forms. When a user uploads an image, it is passed to the OCR module 280 for processing. The OCR module 280 extracts text from the image and converts it into machine-readable data, which is then stored in the platform's database 142, thereby associating it with the corresponding user's health records. The data integration module 230 is coupled to the OCR module 280 to merge the OCR-extracted data with existing user health data, thereby providing a comprehensive health profile.

Additionally, the platform 100 includes a dental Internet of Things (IoT) module 290 configured to collect data from dental IoT devices 172, such as smart toothbrushes and oral hygiene monitors. The platform 100 establishes connectivity with dental IoT devices 172 using relevant communication protocols (e.g., Bluetooth, Wi-Fi). A data ingestion pipeline is developed to collect data from the connected dental IoT devices 172. The dental IoT module 290 implements data processing and analysis algorithms to identify trends and potential dental health issues. The AI engine 260 and machine learning models are coupled to the dental IoT module 290 to generate personalized recommendations for improving dental hygiene based on the analyzed data. The dental IoT module 290 is integrated with the recommendation system 232 to deliver the personalized recommendations to users via the user interface 180.

According to an embodiment, the data integration module 230 further comprises an AI-enhanced questionnaire system 235 that dynamically generates personalized questionnaires based on user profile information and previous responses. A dynamic questionnaire generator is developed within the data integration module 230, wherein said generator utilizes user profile information and previous responses stored in the platform's database 142 to generate personalized questionnaires. Machine learning algorithms (e.g., natural language processing, sentiment analysis) are implemented to analyze user responses and extract insights and patterns. These insights and patterns are used to adapt the questionnaire content and structure in real-time based on user input. The user responses and generated insights are stored in the platform's database 142 for further analysis and integration with other health data.

To support the continuous development and deployment of machine learning models, the digital health self-assessment platform 100 includes a machine learning operations (MLOps) module 255, as shown in FIG. 4. The MLOps module 255 implements a version control system (e.g., Git) to manage the codebase and datasets associated with the platform's machine learning models. An automated pipeline for model deployment is developed, comprising steps such as data preprocessing, model training, and model serving. Monitoring and logging mechanisms are integrated to track the performance of deployed models in real-time. Alerts and triggers are set up to notify the relevant team members when model performance degrades or anomalies are detected. An automated retraining process is implemented, thereby triggering when necessary, based on predefined criteria or scheduled intervals. A user interface is developed for data scientists and ML engineers to collaborate on model development, testing, and deployment.

In some embodiments the OCR module 280 interacts with the user interface 180 to receive uploaded images and with the database 142 to store extracted data. It is also coupled to the data integration module 230 to merge OCR data with existing health records. The dental IoT module 290 interacts with the connected dental IoT devices 172 to collect data, with the AI engine 260 and recommendation system 232 to generate personalized recommendations, and with the user interface 180 to display the recommendations to users. The AI-enhanced questionnaire system 235 interacts with the user interface 180 to present personalized questionnaires, with the database 142 to store user responses and insights, and with the data integration module 230 to integrate questionnaire data with other health data. The MLOps module 255 interacts with the version control system to manage the codebase and datasets, with the deployment pipeline to automate model deployment, and with the monitoring and alerting systems to ensure optimal model performance. It also provides a user interface for collaboration among data scientists and ML engineers.

By incorporating these additional modules and functionalities, the present invention further enhances the capabilities of the digital health self-assessment platform 100 in data processing, oral health management, personalized data collection, and machine learning operations. The OCR module 280 streamlines the integration of medical documents, while the dental IoT module 290 enables the platform to offer comprehensive oral health insights. The AI-enhanced questionnaire system 235 ensures the collection of highly relevant user data, and the MLOps module 255 facilitates the efficient development and deployment of machine learning models. As such, these components work in synergy with the existing modules to provide a holistic and cutting-edge solution for digital health management.

In some embodiment the various components and modules within the digital health self-assessment platform 100 interact with each other through a combination of APIs, data exchange protocols, and event-driven communication. Each module exposes relevant APIs and webhooks to facilitate data sharing and trigger actions across the platform. The user interface 180 interacts with the backend modules using RESTful APIs and WebSocket connections for real-time updates. The data integration module 230 employs various data exchange formats (e.g., JSON, XML) and protocols (e.g., HTTP, MQTT) to collect and integrate data from diverse sources. The AI engine 260 and MLOps module 255 utilize gRPC and protocol buffers for efficient communication between microservices. The recommendation system 232 and other analytics modules consume data from the database 142 and publish insights through message queues (e.g., Apache Kafka) for seamless integration with other components. Asynchronous communication patterns, such as pub/sub and message queues, are used to decouple modules and ensure scalability. By leveraging these interaction methods, the digital health self-assessment platform 100 enables seamless data flow, real-time collaboration, and modular extensibility across its diverse components and functionalities.

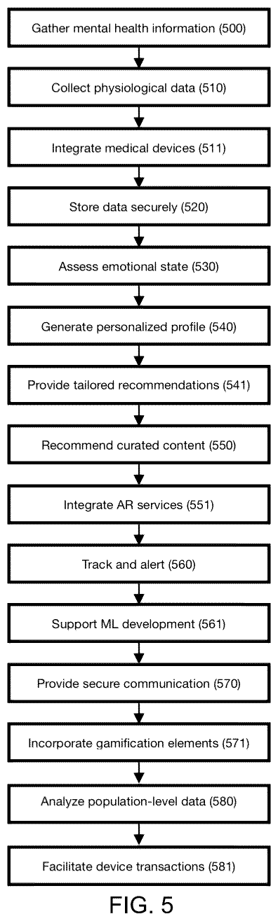

FIG. 5 illustrates an embodiment of a flow diagram of the computer-implemented method for an AI-driven mental health assessment and recommendation using the integrated platform architecture 100. The user interface 180 may include an interactive chatbot 280 that engages in natural language dialogue with the user to gather mental health information 500.

Mental health data is collected from a plurality of sources, including the user input, electronic health records 152, social media, and data from wearable devices 170 such as smartwatches and fitness trackers. The wearables 170 collect physiological data like sleep patterns, physical activity levels, and heart rate variability, wherein said data is analyzed as indicators of mental well-being 510. The method further comprises integrating with and managing wearable and non-wearable medical devices 170 using a device management module 215, receiving data from the medical devices 110, and transmitting control signals to the medical devices 110 511.

The collected multi-modal data is sent via a smartphone or computer 130 to a secure cloud database 142 for storage and use by cloud applications 144 520. A data integration module 230 processes and analyzes the data using a combination of NLP 250, computer vision 240, and machine learning techniques, thereby assessing the user's emotional state and mental well-being 430.

The NLP module 250 performs sentiment analysis on the user's social media posts and chatbot conversations to gauge emotional well-being. The computer vision module 240 analyzes the user's facial expressions and eye movements during video consultations, thereby providing real-time feedback on emotional state.

The processed data is filtered through an individual data filter to generate user-specific insights and a group data filter to identify population-level trends. An AI engine 260 generates a personalized mental health profile comprising a predicted risk level for specific disorders, identified concerns, risk factors, and coping mechanisms 540. The method further comprises providing profile-based recommendations, AI-based translation, health risk measurement, alerting systems, and smart enrollments based on user preferences and historical data using a recommendation system 232 within the AI engine 260 541.

Based on the user's profile, the recommender system 232 provides tailored recommendations for mental health interventions, resources, and support. A library of curated mental health content is recommended to the user via interactive data visualizations 290 and progress is tracked 550. The method further comprises utilizing token-based API requests for enhanced security and encryption, integrating advanced data ingestion and analysis for actionable insights, and providing augmented reality (AR) services 236 for an immersive and interactive user experience, wherein the AR services 236 are configured to superimpose virtual information onto the user's view of the real world 551.

The user's mental health score is calculated based on their profile and tracked over time, with alerts sent to healthcare providers if it drops below a threshold. Regular check-ins and follow-up assessments are scheduled, and reminder notifications are integrated with the user's calendar 560. The method further comprises supporting the development, deployment, and management of machine learning models using a comprehensive data science platform 238, featuring ML model management, a recommendation system 232, detection analysis, and integration with Jupyter Notebook and JupyterLab within the data science platform 238, and enabling data scientists to collaboratively develop and deploy machine learning models using the data science platform 238 561.

Secure communication channels 300 are provided between the user, AI chatbot 280, and mental health professionals for personalized care, with real-time AI-assisted analysis of the conversations. Gamification elements 310 are incorporated, thereby incentivizing engagement and treatment adherence 570. The method further comprises aggregating outputs from multiple artificial intelligence (AI) 260 and machine learning (ML) models, including third-party tools and external databases, using a pooling system 240, providing more robust and reliable outcomes based on the aggregated outputs, and processing and extracting insights from the aggregated outputs using unique algorithms within the pooling system 240 571.

At a population level, the anonymized and aggregated mental health data is analyzed to improve the AI models, identify trends, and provide insights to healthcare providers and policymakers, thereby informing mental health initiatives 580. The method further comprises facilitating the buying and selling of devices and maintenance tickets using a blockchain-based platform 242, featuring a marketplace module, blockchain ledger, smart contracts, user authentication, and a user interface within the blockchain-based platform 242, securely recording transactions using the blockchain ledger, and automating the buying and selling process using the smart contracts 581.

The embodiments described herein are given for the purpose of facilitating the understanding of the present invention and are not intended to limit the interpretation of the present invention. The respective elements and their arrangements, materials, conditions, shapes, sizes, or the like of the embodiment are not limited to the illustrated examples but may be appropriately changed. Further, the constituents described in the embodiment may be partially replaced or combined together.

What is claimed is:

1. A digital health self-assessment platform comprising:
one or more processors;
one or more smart retainer wearable devices equipped with oral-specific sensors and general physiological sensors, wherein the wearables are customizable and distributed to users based on their individual oral composition and the one or more wearable devices collect data points including a unique identifier (ID), pressure measurements, pressure area, mouth acidity levels, accelerometer data, GPS coordinates, temperature readings, device type and ID, and timestamps, wherein the oral-specific sensors comprise dental YLM sensors and oral YLM guards adapted to collect data on tooth positioning and teeth friction pressure;
a memory storing instructions that, when executed by the one or more processors, cause the platform to:
provide a user interface configured to receive user input and display personalized health information;
implement a data integration module configured to collect and store health data from a plurality of sources, including user input, medical devices, and electronic health records;
implement a computer vision module configured to analyze medical images and videos using object recognition, feature extraction, and image segmentation techniques to identify abnormalities and assist in diagnosis;
implement a natural language processing (NLP) module configured to interpret and extract insights from user input, medical records, and questionnaire responses;
implement an artificial intelligence (AI) engine comprising machine learning models trained on health data to generate personalized health insights, predict disease risks, and provide tailored recommendations; and
provide an application programming interface (API) configured to enable integration with third-party applications and services;
implement a dental Internet of Things (IoT) module configured to collect data from dental IoT devices comprising smart toothbrushes and oral hygiene monitors, wherein the platform establishes connectivity with dental IoT devices using communication protocols and a data ingestion pipeline is developed to collect data from the connected dental IoT devices;
wherein the instructions further cause the platform to:
identify trends and potential dental health issues by an AI engine and machine learning models are coupled to the dental IoT module to generate personalized recommendations for improving dental hygiene based on the analyzed data;
generate a personal health score based on the collected health data and AI-generated insights;
provide personalized alerts, notifications, and recommendations to users based on their health status and predicted risks; and
facilitate communication between users and healthcare providers for improved patient-centered care.

2. (canceled)

3. The digital health self-assessment platform of claim 1, wherein the NLP module is further configured to analyze sentiment and emotion in user input to assess mental well-being and provide personalized recommendations for stress management and mental health support.

4. The digital health self-assessment platform of claim 1, wherein the AI engine further comprises a predictive analytics module that utilizes machine learning algorithms to forecast potential health risks and enable early interventions based on user health data and trends.

5. The digital health self-assessment platform of claim 1, wherein the user interface is further configured to present health insights and recommendations using interactive data visualizations, including charts, graphs, and infographics, to facilitate user understanding and engagement.

6. The digital health self-assessment platform of claim 1, further comprising a secure messaging system that enables encrypted communication between users and healthcare providers, allowing for the exchange of health data, test results, and treatment plans.

7. The digital health self-assessment platform of claim 1, further comprising a device management platform that utilizes token-based API requests for enhanced security and encryption, integrates advanced data ingestion and analysis for actionable insights, and provides augmented reality (AR) services for an immersive and interactive user experience, wherein the AR services are configured to superimpose virtual information onto the user's view of the real world.

8. The digital health self-assessment platform of claim 1, further comprising a comprehensive data science platform that supports the development, deployment, and management of machine learning models, featuring ML model management, a recommendation system, detection analysis, and integration with Jupyter Notebook and JupyterLab, wherein the data science platform is configured to enable data scientists to collaboratively develop and deploy machine learning models.

9. The digital health self-assessment platform of claim 1, further comprising a pooling system configured to aggregate outputs from multiple AI and ML models, including third-party tools and external databases, to provide more robust and reliable outcomes, wherein the pooling system comprises unique algorithms to process and extract insights from the aggregated outputs.

10. The digital health self-assessment platform of claim 1, further comprising a blockchain-based platform for buying and selling devices and maintenance tickets, featuring a marketplace module, blockchain ledger, smart contracts, user authentication, and a user interface, wherein the blockchain ledger is configured to securely record transactions and the smart contracts are configured to automate the buying and selling process.

11-20. (canceled)
